(19) United States
(12) Patent Application Publication
(10) Pub. No.: US 2025/0075179 A1
(43) Pub. Date: Mar. 6, 2025

(54) METHODS FOR INCREASING VIRAL TRANSDUCTION OF CELLS

(71) Applicant: Trustees of Dartmouth College, Hanover, NH (US)

(72) Inventors: Margaret Clark CARPENTER, Hanover, NH (US); Margaret E. ACKERMAN, Hanover, NH (US); Yina H. HUANG, Hanover, NH (US)

(21) Appl. No.: 18/263,471

(22) PCT Filed: Jan. 28, 2022
(86) PCT No.: PCT/US2022/014369

§ 371 Date: Jul. 28, 2023

Related U.S. Application Data
(60) Provisional application No. 63/143,286, filed on Jan. 29, 2021.

(51) Int. Cl. C12N 5/0781; A61K 35/17; C12N 15/86

(52) U.S. Cl. CPC C12N 5/0635; A61K 35/17; C12N 15/86; C12N 2501/2302; C12N 2501/2304; C12N 2501/2321; C12N 2501/25; C12N 2501/71; C12N 2501/727; C12N 2501/999; C12N 2740/15043

(57) ABSTRACT

Provided herein are compositions and methods for increasing transduction efficiency of cells (e.g., immune cells) with a viral vector by incubating said cells with one or more agents (e.g., AKT inhibitors and stains such as rosuvastatin) that increase transduction efficiency of cells.

### RELATED APPLICATIONS

This application claims the benefit of U.S. Provisional Application No. 63/143,286, filed Jan. 29, 2021, the entire disclosure of which is hereby incorporated by reference.

### STATEMENT OF FEDERALLY SPONSORED RESEARCH

This invention was made with government support under Grant No. 5R01AI131975-03 awarded by the National Institutes of Health. The Government has certain rights in this invention.

### FIELD OF THE INVENTION

This disclosure relates to compositions and methods of increasing viral transduction of cells (e.g., immune cells).

### BACKGROUND

Human cells, include immune cells, such as T cells, B cells, plasmablasts, and Natural Killer (NK) cells, can be difficult to transduce with a viral vector. These immune cells offer numerous therapeutic benefits, particularly if they can be engineered to confer enhanced or alternative activities. For example, T cells can be engineered to express a target specific TCR or chimeric antigen receptor (CAR) on their surface for target cell killing. B cells can be engineered to secreted specific monoclonal antibodies with therapeutic benefit. One powerful way to engineer cells is through transduction with viral vectors that express proteins or nucleic acids of interest. Unfortunately, low transduction efficiency in these cell types makes engineering said cells for therapeutic benefit costly and time-consuming.

Accordingly, there exists a need to develop methods to enhance the transduction efficiency of cells, such as T cells, B cells, plasmablasts, and NK cells, in vivo, ex vivo, and in vitro, through the use of agents that enhance transduction efficiency.

### SUMMARY

Disclosed herein are compositions and methods of increasing viral transduction of cells.

In one aspect, the disclosure provides a method of increasing transduction efficiency of cells with a viral vector, comprising incubating the cells with one or more transduction efficiency enhancing agents for a sufficient amount of time to increase the transduction efficiency of cells with the viral vector.

In certain embodiments, the cells are immune cells.

In certain embodiments, the cells are selected from the group consisting of: T cells, B cells, plasmablasts, Natural Killer (NK) cells, macrophages, and dendritic cells.

In certain embodiments, the cells are primary cells.

In certain embodiments, the primary cells are human naïve B cells.

In certain embodiments, the human naïve B cells are isolated from peripheral blood mononuclear cells (PBMCs).

In certain embodiments, the primary cells are co-incubated with feeder cells prior to or simultaneously with the one or more transduction efficiency enhancing agents.

In certain embodiments, the feeder cells are stromal feeder cells that express one or both of a cytokine and a CD40 ligand (CD40L).

In certain embodiments, the stromal feeder cells express one or more cytokines.

In certain embodiments, the stromal feeder cells express IL-2. In certain embodiments, the stromal feeder cells express IL-21. In certain embodiments, the stromal feeder cells express IL-2 and IL-21. In certain embodiments, the stromal feeder cells express IL-2, IL-21, and CD40L.

In certain embodiments, the primary cells are isolated from the feeder cells prior to transduction with the viral vector.

In certain embodiments, the primary cells are co-incubated with isolated CD40L prior to or simultaneously with the one or more transduction efficiency enhancing agents.

In certain embodiments, the primary cells are co-incubated with one or more cytokines prior to or simultaneously with the one or more transduction efficiency enhancing agents.

In certain embodiments, the one or more cytokines comprise IL-2, IL-4, IL-21, B-cell activating factor (BAFF), or a combination thereof.

In certain embodiments, the primary cells are co-incubated with: IL-2 at a concentration of about 5 ng/mL to about 100 ng/mL; IL-4 at a concentration of about 0.1 ng/mL to about 50 ng/mL; IL-21 at a concentration of about 0.1 ng/mL to about 50 ng/mL; and/or BAFF at a concentration of about 0.1 ng/mL to about 50 ng/mL.

In certain embodiments, the primary cells are co-incubated with: IL-2 at a concentration of about 50 ng/mL; IL-4 at a concentration of about 10 ng/mL; IL-21 at a concentration of about 10 ng/mL; and/or BAFF at a concentration of about 10 ng/mL.

In certain embodiments, the cells retain activity after incubation with the one or more transduction efficiency enhancing agents.

In certain embodiments, the cells retain the ability to produce antibodies after incubation with the one or more transduction efficiency enhancing agents.

In certain embodiments, the cells retain antibody class switching activity after incubation with the one or more transduction efficiency enhancing agents.

In certain embodiments, the cells retain cytotoxic activity after incubation with the one or more transduction efficiency enhancing agents.

In certain embodiments, the cells retain at least about 20%, at least about 30%, at least about 40%, at least about 50%, at least about 60%, at least about 70%, at least about 80%, at least about 90%, or at least about 95% cytotoxic activity after incubation with the one or more agents relative to cells that are not incubated with the one or more transduction efficiency enhancing agents.

In certain embodiments, the one or more transduction efficiency enhancing agents increase expression of LDL receptor (LDLR) on the cells.

In certain embodiments, the one or more transduction efficiency enhancing agents increase expression of LDLR on the cells by about 1.5-fold to about 100-fold in comparison to cells in the absence of the one or more agents.

In certain embodiments, the one or more transduction efficiency enhancing agents increase expression of LDLR on the cells by about 1.5-fold, about 2-fold, about 3-fold, about 4-fold, about 5-fold, about 10-fold, about 20-fold, about 30-fold, about 40-fold, about 50-fold, about 60-fold, about 70-fold, about 80-fold, about 90-fold, or about 100-fold in comparison to cells in the absence of the one or more agents.

In certain embodiments, the one or more transduction efficiency enhancing agents is an AKT inhibitor.

In certain embodiments, the AKT inhibitor is an ATP-competitive inhibitor or an allosteric inhibitor.

In certain embodiments, the AKT inhibitor comprises ARQ 092, ARQ 751, AT7867, AT13148, A-674563, BAY1125976, capivasertib (also known as AZD5363), GSK690693, GSK2110183, ipatasertib (also known as GDC-0068), LY2780301, miransertib, MK-2206, PF-04691502, triciribine, or a combination thereof.

In certain embodiments, the one or more transduction efficiency enhancing agents is a statin.

In certain embodiments, the statin comprises rosuvastatin, atorvastatin, fluvastatin, lovastatin, pitavastatin, pravastatin, simvastatin, or a combination thereof.

In certain embodiments, the cells are incubated with the one or more statins at a concentration of about 0.1 μM to about 100 μM.

In certain embodiments, the cells are incubated with the one or more statins at a concentration of about 0.5 μM, about 1 μM, about 5 μM, about 10 μM, about 15 μM, about 20 RM, about 25 μM, about 30 μM, about 35 μM, about 40 μM, about 45 μM, about 50 μM, about 75 μM, or about 100 μM.

In certain embodiments, the transduction is carried out in vivo.

In certain embodiments, the transduction is carried out ex vivo or in vitro.

In certain embodiments, at least about 20% (i.e., about 20%, about 25%, about 30%, about 35%, about 40%, about 45%, about 50%, about 55%, about 60%, about 65%, about 70%, about 75%, about 80%, about 85%, about 90%, or about 95%) of the cells are transduced with the viral vector after incubation with the one or more transduction efficiency enhancing agents.

In certain embodiments, at least about 50% of the cells are transduced with the viral vector after incubation with the one or more transduction efficiency enhancing agents.

In certain embodiments, at least about 75% of the cells are transduced with the viral vector after incubation with the one or more transduction efficiency enhancing agents.

In certain embodiments, at least about 90% of the cells are transduced with the viral vector after incubation with the one or more transduction efficiency enhancing agents.

In certain embodiments, the transduction efficiency is increased about 2-fold to about 300-fold in comparison to transduction in the absence of the one or more transduction efficiency enhancing agents.

In certain embodiments, the transduction efficiency is increased about 2-fold, about 5-fold, about 10-fold, about 20-fold, about 30-fold, about 40-fold, about 50-fold, about 60-fold, about 70-fold, about 80-fold, about 90-fold, or about 100-fold in comparison to transduction in the absence of the one or more transduction efficiency enhancing agents.

In certain embodiments, the viral vector is a lentiviral vector or a retroviral vector.

In certain embodiments, the lentiviral vector is a Human immunodeficiency virus (HIV) virus.

In certain embodiments, the viral vector is pseudotyped with a vesicular stomatitis virus G-protein (VSV-G) envelope protein.

In another aspect, the disclosure provides a population of cells prepared according to the methods described above.

In another aspect, the disclosure provides a pharmaceutical composition comprising the population of cells described above.

In one aspect, the disclosure provides a method of transducing a population of cells comprising the steps of: a) contacting the population of cells with one or more transduction efficiency enhancing agents; and b) transducing the population of cells with a viral vector; wherein transduction efficiency is increased in comparison to transduction in the absence of the one or more transduction efficiency enhancing agents.

In certain embodiments, the cells are immune cells.

In certain embodiments, the cells are selected from the group consisting of: T cells, B cells, plasmablasts, Natural Killer (NK) cells, macrophages, and dendritic cells.

In certain embodiments, the cells are primary cells.

In certain embodiments, the primary cells are human naïve B cells.

In certain embodiments, the human naïve B cells are isolated from peripheral blood mononuclear cells (PBMCs).

In certain embodiments, the primary cells are co-incubated with feeder cells prior to or simultaneously with the one or more transduction efficiency enhancing agents.

In certain embodiments, the feeder cells are stromal feeder cells that express one or both of a cytokine and a CD40 ligand (CD40L).

In certain embodiments, the stromal feeder cells express one or more cytokines.

In certain embodiments, the stromal feeder cells express IL-2. In certain embodiments, the stromal feeder cells express IL-21. In certain embodiments, the stromal feeder cells express IL-2 and IL-21. In certain embodiments, the stromal feeder cells express IL-2, IL-21, and CD40L.

In certain embodiments, the primary cells are isolated from the feeder cells prior to transduction with the viral vector.

In certain embodiments, the primary cells are co-incubated with one or more cytokines prior to or simultaneously with the one or more transduction efficiency enhancing agents.

In certain embodiments, the one or more cytokines comprise IL-2, IL-4, IL-21, B-cell activating factor (BAFF), or a combination thereof.

In certain embodiments, the primary cells are co-incubated with: IL-2 at a concentration of about 5 ng/mL to about 100 ng/mL; IL-4 at a concentration of about 0.1 ng/mL to about 50 ng/mL; IL-21 at a concentration of about 0.1 ng/mL to about 50 ng/mL; and/or BAFF at a concentration of about 0.1 ng/mL to about 50 ng/mL.

In certain embodiments, the primary cells are co-incubated with: IL-2 at a concentration of about 50 ng/mL; IL-4 at a concentration of about 10 ng/mL; IL-21 at a concentration of about 10 ng/mL; and/or BAFF at a concentration of about 10 ng/mL.

In certain embodiments, the cells retain activity after incubation with the one or more transduction efficiency enhancing agents.

In certain embodiments, the cells retain the ability to produce antibodies after incubation with the one or more transduction efficiency enhancing agents.

In certain embodiments, the cells retain antibody class switching activity after incubation with the one or more transduction efficiency enhancing agents.

In certain embodiments, the cells retain cytotoxic activity after incubation with the one or more transduction efficiency enhancing agents.

In certain embodiments, the cells retain at least about 20%, at least about 30%, at least about 40%, at least about 50%, at least about 60%, at least about 70%, at least about 80%, at least about 90%, or at least about 95% cytotoxic activity after incubation with the one or more agents relative to cells that are not incubated with the one or more transduction efficiency enhancing agents.

In certain embodiments, the one or more transduction efficiency enhancing agents increase expression of LDL receptor (LDLR) on the cells.

In certain embodiments, the one or more transduction efficiency enhancing agents increase expression of LDLR on the cells by about 1.5-fold to about 100-fold in comparison to cells in the absence of the one or more agents.

In certain embodiments, the one or more transduction efficiency enhancing agents increase expression of LDLR on the cells by about 1.5-fold, about 2-fold, about 3-fold, about 4-fold, about 5-fold, about 10-fold, about 20-fold, about 30-fold, about 40-fold, about 50-fold, about 60-fold, about 70-fold, about 80-fold, about 90-fold, or about 100-fold in comparison to cells in the absence of the one or more agents.

In certain embodiments, the transduction efficiency enhancing agents is an AKT inhibitor.

In certain embodiments, the AKT inhibitor is an ATP-competitive inhibitor or an allosteric inhibitor.

In certain embodiments, the AKT inhibitor comprises ARQ 092, ARQ 751, AT7867, AT13148, A-674563, BAY1125976, capivasertib (also known as AZD5363), GSK690693, GSK2110183, ipatasertib (also known as GDC-0068), LY2780301, miransertib, MK-2206, PF-04691502, triciribine, or a combination thereof.

In certain embodiments, the transduction efficiency enhancing agents is a statin.

In certain embodiments, the statin comprises rosuvastatin, atorvastatin, fluvastatin, lovastatin, pitavastatin, pravastatin, simvastatin, or a combination thereof.

In certain embodiments, the cells are incubated with the one or more statins at a concentration of about 0.1 μM to about 100 μM.

In certain embodiments, the cells are incubated with the one or more statins at a concentration of about 0.5 μM, about 1 μM, about 5 μM, about 10 μM, about 15 μM, about 20 RM, about 25 μM, about 30 μM, about 35 μM, about 40 μM, about 45 μM, about 50 μM, about 75 μM, or about 100 μM.

In certain embodiments, steps (a) and (b) are carried out in vivo.

In certain embodiments, steps (a) and (b) are carried out ex vivo or in vitro.

In certain embodiments, steps (a) and (b) are performed simultaneously or sequentially.

In certain embodiments, at least about 20% (i.e., about 20%, about 25%, about 30%, about 35%, about 40%, about 45%, about 50%, about 55%, about 60%, about 65%, about 70%, about 75%, about 80%, about 85%, about 90%, or about 95%) of the cells are transduced with the viral vector after incubation with the one or more transduction efficiency enhancing agents.

In certain embodiments, at least about 50% of the cells are transduced with the viral vector after incubation with the one or more transduction efficiency enhancing agents.

In certain embodiments, at least about 75% of the cells are transduced with the viral vector after incubation with the one or more transduction efficiency enhancing agents.

In certain embodiments, at least about 90% of the cells are transduced with the viral vector after incubation with the one or more transduction efficiency enhancing agents.

In certain embodiments, the transduction efficiency is increased about 2-fold to about 300-fold in comparison to transduction in the absence of the one or more transduction efficiency enhancing agents.

In certain embodiments, the transduction efficiency is increased about 2-fold, about 5-fold, about 10-fold, about 20-fold, about 30-fold, about 40-fold, about 50-fold, about 60-fold, about 70-fold, about 80-fold, about 90-fold, or about 100-fold in comparison to transduction in the absence of the one or more transduction efficiency enhancing agents.

In certain embodiments, the viral vector is a lentiviral vector or a retroviral vector.

In certain embodiments, the lentiviral vector is a Human immunodeficiency virus (HIV) virus.

In certain embodiments, the viral vector is pseudotyped with a vesicular stomatitis virus G-protein (VSV-G) envelope protein.

In another aspect, the disclosure provides a population of cells prepared according to the methods described above.

In another aspect, the disclosure provides a pharmaceutical composition comprising the population of cells described above.

In one aspect, the disclosure provides a method of transducing a population of primary B cells comprising the steps of: a) contacting the population of primary B cells with one or more statins; and b) transducing the population of primary B cells with a lentiviral vector; wherein transduction efficiency is increased in comparison to transduction in the absence of the one or more statins.

In certain embodiments, the transduction efficiency is increased about 2-fold to about 300-fold in comparison to transduction in the absence of the one or more statins.

In certain embodiments, the transduction efficiency is increased about 2-fold, about 5-fold, about 10-fold, about 20-fold, about 30-fold, about 40-fold, about 50-fold, about 60-fold, about 70-fold, about 80-fold, about 90-fold, or about 100-fold in comparison to transduction in the absence of the one or more statins.

In certain embodiments, the primary B cells are human naïve B cells.

In certain embodiments, the human naïve B cells are isolated from peripheral blood mononuclear cells (PBMCs).

In certain embodiments, the primary B cells are human naïve B cells; the statin comprises rosuvastatin at a concentration of about 0.5 μM to about 50 μM; and the lentiviral vector is pseudotyped with a VSV-G envelope protein.

In certain embodiments, the transduction efficiency is increased about 100-fold in comparison to transduction in the absence of rosuvastatin.

In one aspect, the disclosure provides a composition comprising a population of primary cells, one or more statins, and a population of feeder cells.

In certain embodiments, the composition further comprises one or more LDL-R inhibitors. In certain embodiments, the LDL-R inhibitor comprises an anti-LDL-R antibody or fragment thereof.

In certain embodiments, the feeder cells are stromal feeder cells that express one or both of a cytokine and a CD40 ligand (CD40L). In certain embodiments, the stromal feeder cells express one or more cytokines. In certain embodiments, the stromal feeder cells express IL-2 and IL-21. In certain embodiments, the stromal feeder cells express IL-2, IL-21, and CD40L.

### BRIEF DESCRIPTION OF THE DRAWINGS

The foregoing and other features and advantages of the present invention will be more fully understood from the following detailed description of illustrative embodiments taken in conjunction with the accompanying drawings. The patent or application file contains at least one drawing executed in color. Copies of this patent or patent application publication with color drawing(s) will be provided by the Office upon request and payment of the necessary fee.

FIG. 1A-FIG. 1C graphically depict a schematic B cell transduction efficiency by flow cytometry. B cells were cultured in the absence (FIG. 1A) or presence of a statin with recombinant CD40L (FIG. 1B) for 48 hours prior to viral transduction or presence of statin with CD40L expressing feeder cells (FIG. 1B) for 48 hours prior to viral transduction. Flow cytometry was used to detect lentiviral transduction of green fluorescent protein (GFP). In FIGS. 1B and C, one infection was performed with lentivirus that did not encode GFP as a control for GFP expression (GFP-lentivirus, shown in dark grey). Five percent of cells culture without statin express GFP 48 hours after lentiviral transduction (FIGS. 1A and B, light grey), whereas at least eighty percent of cells cultured with statin and CD40L expressing feeder cells expressed GFP, regardless of statin dose (FIG. 1C, lighter greys).

FIG. 2 graphically depicts percent B cells expressing GFP after transduction with a lentivirus expressing GFP. Each dot represents a technical replicate of the transduction procedure, mean value indicated by bars. After treatment with a statin or PBS control, cells were transduced with lentivirus to express GFP (GFP+) or control lentivirus that does not encode GFP (GFP−). Student t-testing indicates significant difference between conditions (*** indicates p-value less than 0.0005, **** indicates p-value less than 0.0001).

FIG. 3 graphically depicts naïve B cell antibody class switching in statin and PBS treated cells.

FIG. 4 graphically depicts a schematic B cell transduction efficiency by flow cytometry. B cells were co-cultured with feeder cells expressing CD40L, followed by incubation with a GFP-expressing lentivirus.

### DETAILED DESCRIPTION

Compositions and methods of increasing viral transduction of cells (e.g., immune cells) are provided.

In one aspect, the disclosure provides a method of increasing transduction efficiency of cells (e.g., immune cells) with a viral vector, comprising incubating the cells with one or more transduction efficiency enhancing agents for a sufficient amount of time to increase the transduction efficiency of cells with the viral vector.

In one aspect, the disclosure provides a method of increasing transduction efficiency of cells (e.g., immune cells) with a viral vector, comprising incubating the cells with one or more statins for a sufficient amount of time to increase the transduction efficiency of cells with the viral vector.

In one aspect, the disclosure provides a method of increasing transduction efficiency of cells (e.g., immune cells) with a viral vector, comprising incubating the cells with one or more AKT inhibitors for a sufficient amount of time to increase the transduction efficiency of cells with the viral vector.

In another aspect, the disclosure provides a method of transducing a population of cells (e.g., immune cells) comprising the steps of: a) contacting the population of cells with one or more transduction efficiency enhancing agents; and b) transducing the population of cells with a viral vector; wherein transduction efficiency is increased in comparison to transduction in the absence of the one or more transduction efficiency enhancing agents.

In a further aspect, the disclosure provides a method of transducing a population of cells (e.g., immune cells) comprising the steps of: a) contacting the population of cells with one or more AKT inhibitors; and b) transducing the population of cells with a lentiviral vector; wherein transduction efficiency is increased in comparison to transduction in the absence of the one or more AKT inhibitors.

In yet another aspect, the disclosure provides a method of transducing a population of primary B cells comprising the steps of: a) contacting the population of primary B cells with one or more statins; and b) transducing the population of primary B cells with a lentiviral vector; wherein transduction efficiency is increased in comparison to transduction in the absence of the one or more statins.

The disclosure further provides a population of cells prepared according to the methods described above.

The disclosure further provides a pharmaceutical composition comprising the population of cells recited above.

Generally, nomenclature used in connection with cell and tissue culture, molecular biology, immunology, microbiology, genetics and protein and nucleic acid chemistry and hybridization described herein are those well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

### Definitions

As used here, the terms “transduction efficiency enhancing agent” or “transduction efficiency increasing agent” or “transduction efficiency promoting agent” or simply “agent” refer to compounds that increase the transduction efficiency of a target cell (or population of cells) to be transduced with a viral vector. The agents of the disclosure can increase transduction efficiency through a variety of mechanisms, including, but not limited to, increasing expression of the LDL receptor (LDLR) on the target cells.

In certain embodiments, the transduction efficiency enhancing agent is a HMG-CoA reductase inhibitor, e.g., a statin. Non-limiting examples of statins include rosuvastatin, atorvastatin, fluvastatin, lovastatin, pitavastatin, pravastatin, simvastatin, or a combination thereof.

In certain embodiments, the transduction efficiency enhancing agent is an AKT (i.e., protein kinase B, PKB) inhibitor. The AKT inhibitor can be an ATP-competitive AKT inhibitor or an allosteric AKT inhibitor. Moreover, the inhibitors can be pan-AKT inhibitors (i.e., inhibiting multiple or all AKT isoforms) or selective AKT inhibitors (i.e., inhibiting a specific AKT isoform, such as AKT1, AKT2, or AKT3). ATP-competitive inhibitors target the catalytic site of the active kinase in the PH-out conformation and prevent substrate phosphorylation (see, Lin et al. Sci. Signal. 5, ra37 10.1126/scisignal.2002618. 2012; Addie et al. J. Med. Chem. 56, 2059-2073 10.1021/jm301762v. 2013). Allosteric inhibitors target an allosteric pocket within the PH-domain/kinase-domain interface of AKT that stabilizes the PH-in conformation. Allosteric AKT inhibitors, lock AKT in an auto-inhibited conformation and interfere with PH-domain mediated-membrane recruitment, thus preventing AKT kinase activation and AKT phosphorylation (see, Calleja et al. PLoS Biol. 5, 780-791 10.1371/journal.pbio.0050095. 2007; Calleja et al. J. Chem. Biol. 2, 11-25 10.1007/s12154-009-0016-8. 2009; Wu et al. PLoS One 5, e1291 10.1371/journal.pone.0012913. 2010). Additional details about the different AKT inhibitors, including examples of inhibitors in each class, are described further in Lazaro et al. (Biochem Soc Trans. 48(3): 933-943. 2020).

Non-limiting examples of AKT inhibitors include ARQ 092, ARQ 751, AT7867, AT13148, A-674563, BAY1125976, capivasertib (also known as AZD5363), GSK690693, GSK2110183, ipatasertib (also known as GDC-0068), LY2780301, miransertib, MK-2206, PF-04691502, triciribine, or a combination thereof.

Non-limiting examples of ATP-competitive AKT inhibitors include AT7867, capivasertib (also known as AZD5363), GSK690693, GSK2110183, ipatasertib (also known as GDC-0068), LY2780301, or a combination thereof.

Non-limiting examples of allosteric AKT inhibitors include ARQ 092, ARQ 751, BAY1125976, miransertib, MK-2206, or a combination thereof.

In certain embodiments, the transduction efficiency enhancing agent is a population of stromal feeder cells that express one or both of a cytokine and a CD40 ligand (CD40L).

In certain embodiments, the transduction efficiency enhancing agent is isolated CD40L.

In certain embodiments, the transduction efficiency enhancing agent is an LDL-R inhibitor. The LDL-R inhibitor may be any LDL-R inhibitor known in the art, including small molecule inhibitors and antagonistic anti-LDL-R antibodies or fragments thereof. Exemplary anti-LDL-R antibodies include, but are not limited to, R&D Systems AF2255 antibody, Abcam ab52818 antibody, Santa Cruz Biotechnology F-7 antibody, Invitrogen PA5-46987 antibody, and Novus Biologics NBP1-06709 antibody.

### Transducable Cells

Certain immune cell types can be difficult to transduce with a viral vector due to low transduction efficiency of the target cells. It is therefore advantageous to enhance the transduction efficiency of said cells prior to or during transduction with a viral vector. In certain embodiments, the cell or population of cells to be transduced is an/are immune cell(s). In certain embodiments, the cells are selected from the group consisting of: T cells, B cells, plasmablasts, Natural Killer (NK) cells, macrophages, and dendritic cells. In certain embodiments, the cells are primary cells. In certain embodiments, the primary cells are human naïve B cells, peripheral blood CD56+ NK cells, peripheral blood CD8+ cytotoxic T cells, peripheral blood CD4+ helper T cells, or peripheral blood CD14+ monocytes. The human naïve B cells can be peripheral blood CD19+ B cells.

In certain embodiments, the human naïve B cells are isolated from peripheral blood mononuclear cells (PBMCs).

The primary cells can be treated with compounds or co-incubated with additional cell types to activate said cells, including, but not limited to, differentiation and/or proliferation of said primary cells. For example, but in no way limiting, human naïve B cells can be co-incubated with stromal feeder cells that express one or more B cell differentiation factors, such as CD40 ligand (CD40L). After an incubation time sufficient to induce cell proliferation, the cells can be isolated and transduced with a viral vector. A transduction efficiency enhancing agent can be co-incubated with the primary cells before, during, or after co-incubation with the stromal feeder cells.

In certain embodiments, the primary cells are co-incubated with feeder cells prior to, simultaneously with, or after the one or more transduction efficiency enhancing agents. In certain embodiments, the feeder cells are stromal feeder cells that express one or both of a cytokine and a CD40L. In certain embodiments, the CD40L is membrane-bound CD40L. In certain embodiments, the stromal feeder cells express one or more cytokines. In certain embodiments, the stromal feeder cells express one or both of IL-2 and IL-21. In certain embodiments, the stromal feeder cells express one or more of IL-2, IL-21, and CD40L. In certain embodiments, the stromal feeder cells express IL-2, IL-21, and CD40L. In certain embodiments, the primary cells are isolated from the feeder cells prior to transduction with the viral vector.

In certain embodiments, the primary cells are co-incubated with one or more cytokines prior to, simultaneously with, or after the one or more transduction efficiency enhancing agents. In certain embodiments, the one or more cytokines comprise IL-2, IL-4, IL-21, B-cell activating factor (BAFF), or a combination thereof.

In certain embodiments, the primary cells are co-incubated with: IL-2 at a concentration of about 5 ng/mL to about 100 ng/mL; IL-4 at a concentration of about 0.1 ng/mL to about 50 ng/mL; IL-21 at a concentration of about 0.1 ng/mL to about 50 ng/mL; and/or BAFF at a concentration of about 0.1 ng/mL to about 50 ng/mL.

In certain embodiments, the primary cells are co-incubated with: IL-2 at a concentration of about 50 ng/mL; IL-4 at a concentration of about 10 ng/mL; IL-21 at a concentration of about 10 ng/mL; and/or BAFF at a concentration of about 10 ng/mL.

In certain embodiments, the primary cells are co-incubated with a soluble CD40L prior to, simultaneously with, or after the one or more transduction efficiency enhancing agents.

In certain embodiments, the cells (e.g., B cells, plasmablasts, NK cells, T cells) retain activity after incubation with the one or more transduction efficiency enhancing agents. The activity can be a normal cellular activity of the particular cell type. For example, but in no way limiting, the activity of a B cell can be the ability to produce and secrete antibodies. Retention of an activity after incubation with the one or more transduction efficiency enhancing agents does not require retention of the full (i.e., 100% retention) activity prior to incubation with the one or more transduction efficiency enhancing agents. A sufficient level of activity should be maintained to produce a therapeutically beneficial outcome.

In certain embodiments, the cells (e.g., B cells or plasmablasts) retain the ability to produce antibodies after incubation with the one or more transduction efficiency enhancing agents.

In certain embodiments, the cells (e.g., B cells or plasmablasts) retain antibody class switching activity after incubation with the one or more transduction efficiency enhancing agents. As used herein, the term “antibody class switching” or “immunoglobulin class switching” or “isotype switching” or “isotypic commutation” or “class-switch recombination” or “CSR”, refers to a process that changes a B cell's production of one immunoglobulin isotype to a different immunoglobulin isotype, such as from the isotype IgM to the isotype IgG. During this process, the constant-region portion of the antibody heavy chain is changed, but the variable region of the heavy chain remains unchanged, and the antibody therefore retains antigen specificity. As used herein, the term “antibody class switching activity” refers to the process of antibody class switching as described above. Cells capable of undergoing antibody class switching can retain this ability after incubation with the one or more transduction efficiency enhancing agents.

In certain embodiments, the cells (e.g., NK cells or T cells) retain cytotoxic activity after incubation with the one or more transduction efficiency enhancing agents.

In certain embodiments, the cells (e.g., NK cells or T cells) retain at least about 20%, at least about 30%, at least about 40%, at least about 50%, at least about 60%, at least about 70%, at least about 80%, at least about 90%, or at least about 95% cytotoxic activity after incubation with the one or more agents relative to cells (e.g., NK cells or T cells) that are not incubated with the one or more transduction efficiency enhancing agents.

Cytotoxic activity can be measured using any suitable method known in the art, such as cytotoxicity assays described in, for example, Kochenderfer et al., J. Immunotherapy, 32(7): 689-702 (2009), and Herman et al. J. Immunological Methods, 285(1): 25-40 (2004).

In certain embodiments, the one or more transduction efficiency enhancing agents increase expression of LDL receptor (LDLR) on the cells.

In certain embodiments, the one or more transduction efficiency enhancing agents increase expression of LDLR on the cells by about 1.5-fold to about 100-fold in comparison to cells in the absence of the one or more transduction efficiency enhancing agents.

In certain embodiments, the one or more transduction efficiency enhancing agents increase expression of LDLR on the cells by about 1.5-fold, about 2-fold, about 3-fold, about 4-fold, about 5-fold, about 10-fold, about 20-fold, about 30-fold, about 40-fold, about 50-fold, about 60-fold, about 70-fold, about 80-fold, about 90-fold, or about 100-fold in comparison to cells in the absence of the one or more transduction efficiency enhancing agents.

In certain embodiments, the one or more transduction efficiency enhancing agents increase expression of LDLR on the cells by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 125%, at least about 150%, at least about 175%, at least about 200%, at least about 300%, at least about 400%, at least about 500%, at least about 600%, at least about 700%, at least about 800%, at least about 900%, or at least about 1000%, in comparison to cells in the absence of the one or more transduction efficiency enhancing agents.

In certain embodiments, the cells (e.g., immune cells) can be incubated with the one or more transduction efficiency enhancing agents at a concentration sufficient to increase the transduction efficiency the cells. In certain embodiments, the cells (e.g., immune cells) can be incubated with the one or more transduction efficiency enhancing agents at a concentration sufficient to increase expression of LDLR on the cells.

In certain embodiments, the cells (e.g., immune cells) can be incubated with the one or more transduction efficiency enhancing agents at a concentration of about 0.1 μM to about 100 RM. In certain embodiments, the cells (e.g., immune cells) can be incubated with the one or more transduction efficiency enhancing agents at a concentration of about 0.1 μM to about 10 μM. In certain embodiments, the cells (e.g., immune cells) can be incubated with the one or more transduction efficiency enhancing agents at a concentration of about 0.5 μM to about 20 μM. In certain embodiments, the cells (e.g., immune cells) can be incubated with the one or more transduction efficiency enhancing agents at a concentration of about 0.1 μM, about 0.5 μM, about 1 μM, about 5 μM, about 10 μM, about 15 μM, about 20 μM, about 25 μM, about 30 μM, about 35 μM, about 40 μM, about 45 μM, about 50 μM, about 75 μM, or about 100 μM.

In certain embodiments, the cells (e.g., immune cells) can be incubated with the one or more AKT inhibitors at a concentration of about 0.1 μM to about 100 μM. In certain embodiments, the cells (e.g., immune cells) can be incubated with the one or more AKT inhibitors at a concentration of about 0.1 μM to about 10 μM. In certain embodiments, the cells (e.g., immune cells) can be incubated with the one or more AKT inhibitors at a concentration of about 0.5 μM to about 20 μM. In certain embodiments, the cells (e.g., immune cells) can be incubated with the one or more AKT inhibitors at a concentration of about 0.1 μM, about 0.5 μM, about 1 μM, about 5 μM, about 10 μM, about 15 μM, about 20 μM, about 25 μM, about 30 μM, about 35 μM, about 40 μM, about 45 μM, about 50 μM, about 75 μM, or about 100 μM.

In certain embodiments, the cells (e.g., immune cells) can be incubated with the one or more statins at a concentration of about 0.1 μM to about 100 μM. In certain embodiments, the cells (e.g., immune cells) can be incubated with the one or more statins at a concentration of about 0.1 μM to about 10 μM. In certain embodiments, the cells (e.g., immune cells) can be incubated with the one or more statins at a concentration of about 0.5 μM to about 20 μM. In certain embodiments, the cells (e.g., immune cells) can be incubated with the one or more statins at a concentration of about 0.1 μM, about 0.5 μM, about 1 μM, about 5 μM, about 10 μM, about 15 RM, about 20 μM, about 25 μM, about 30 μM, about 35 μM, about 40 μM, about 45 μM, about 50 μM, about 75 μM, or about 100 μM.

In certain embodiments, the transduction is carried out in vivo. For in vivo transduction, a subject can be administered one or more transduction efficiency enhancing agents for a period of time sufficient to enhance transduction efficiency of one or more cells in the subject, followed by administering a viral vector for in vivo transduction of the one or more cells in the subject.

In certain embodiments, the transduction is carried out ex vivo or in vitro. For ex vivo transduction, cells to be transduced can be isolated from a subject, followed by incubating the isolated cells with one or more transduction efficiency enhancing agents for a period of time sufficient to enhance transduction efficiency of the isolated cells, followed by administering or incubating the cells with a viral vector for ex vivo transduction of the isolated cells.

In certain embodiments, at least about 10%, at least about 20%, at least about 30%, at least about 40%, at least about 50%, at least about 60%, at least about 70%, at least about 80%, at least about 90%, about 95%, about 96%, about 97%, about 98%, about 99%, or 100%, of the cells are transduced with the viral vector after incubation with the one or more transduction efficiency enhancing agents. In certain embodiments, at least about 50% of the cells are transduced with the viral vector after incubation with the one or more transduction efficiency enhancing agents. In certain embodiments, at least about 75% of the cells are transduced with the viral vector after incubation with the one or more transduction efficiency enhancing agents. In certain embodiments, at least about 90% of the cells are transduced with the viral vector after incubation with the one or more transduction efficiency enhancing agents.

In certain embodiments, the transduction efficiency is increased about 2-fold to about 300-fold in comparison to transduction in the absence of the one or more transduction efficiency enhancing agents. In certain embodiments, the transduction efficiency is increased about 2-fold to about 100-fold in comparison to transduction in the absence of the one or more transduction efficiency enhancing agents. In certain embodiments, the transduction efficiency is increased about 2-fold, about 5-fold, about 10-fold, about 20-fold, about 30-fold, about 40-fold, about 50-fold, about 60-fold, about 70-fold, about 80-fold, about 90-fold, or about 100-fold in comparison to transduction in the absence of the one or more transduction efficiency enhancing agents.

In certain embodiments, the transduction efficiency is increased by at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 125%, at least about 150%, at least about 175%, at least about 200%, at least about 300%, at least about 400%, at least about 500%, at least about 600%, at least about 700%, at least about 800%, at least about 900%, or at least about 1000%, in comparison to transduction in the absence of the one or more transduction efficiency enhancing agents.

### Viral Vectors

In certain embodiments, the viral vector is a retroviral vector. “Retroviruses” are enveloped RNA viruses that are capable of infecting animal cells, and that utilize the enzyme reverse transcriptase in the early stages of infection to generate a DNA copy from their RNA genome, which is then typically integrated into the host genome. Non-limiting examples of retroviral vectors are Moloney murine leukemia virus (MLV)-derived vectors, retroviral vectors based on a Murine Stem Cell Virus (see, e.g., Hawley et al., PNAS USA 93:10297-10302, 1996; Keller et al., Blood 92:877-887, 1998), hybrid vectors (see, e.g., Choi, et al., Stem Cells 19:236-246, 2001), and complex retrovirus-derived vectors, such as lentiviral vectors. In certain embodiments the retroviral vector is a murine leukemia virus (MLV) vector.

In certain embodiments, the viral vector is a lentiviral vector. The term “lentivirus” refers to a genus of complex retroviruses that are capable of infecting both dividing and non-dividing cells. Non-limiting examples of lentiviruses include HIV (human immunodeficiency virus; including HIV type 1 and HIV type 2), visna-maedi virus, the caprine arthritis-encephalitis virus, equine infectious anemia virus, feline immunodeficiency virus (FIV), bovine immune deficiency virus (BIV), and simian immunodeficiency virus (SIV). Lentiviral vectors can be derived from any one or more of these lentiviruses (see, e.g., Evans et al., Hum Gene Ther. 10:1479-1489, 1999; Case et al., PNAS USA 96:2988-2993, 1999; Uchida et al., PNAS USA 95:11939-11944, 1998; Miyoshi et al., Science 283:682-686, 1999; Sutton et al., J Virol 72:5781-5788, 1998; and Frecha et al., Blood. 112:4843-52, 2008, each of which is incorporated by reference in its entirety). In certain embodiments, the lentiviral vector is a Human immunodeficiency virus (HIV) vector.

In certain embodiments, the viral vector (e.g., retroviral, lentiviral) is “pseudotyped” with one or more selected viral glycoproteins or envelope proteins, primarily to target selected cell types. Pseudotyping refers generally to the incorporation of one or more heterologous viral glycoproteins onto the cell-surface virus particle, often allowing the virus particle to infect a selected cell that differs from its normal target cells. A “heterologous” element is derived from a virus other than the virus from which the RNA genome of the viral vector is derived. Typically, the glycoprotein-coding regions of the viral vector have been genetically altered such as by deletion to prevent expression of its own glycoprotein. Merely by way of illustration, the envelope glycoproteins gp41 and/or gp120 from an HIV-derived lentiviral vector are typically deleted prior to pseudotyping with a heterologous viral glycoprotein.

In certain embodiments, the viral vector is pseudotyped with a heterologous viral glycoprotein that targets immune cells, such as B cells, plasmablasts, NK cells, or T cells. In certain embodiments, the viral glycoprotein allows selective infection or transduction of B cells. In certain embodiments, the viral glycoprotein allows selective infection or transduction of resting or quiescent B cells. In certain embodiments, the viral glycoprotein allows selective infection or transduction of activated B cells. In certain embodiments, the viral glycoprotein allows infection or transduction of both quiescent B cells and activated B cells. In certain embodiments, the viral glycoprotein allows selective infection or transduction of primary cells. In certain embodiments, the viral glycoprotein allows selective infection or transduction of human naïve B cells. In certain embodiments, the viral glycoprotein allows selective infection or transduction of plasmablasts. In certain embodiments, the viral glycoprotein allows selective infection or transduction of T cells. In certain embodiments, the viral glycoprotein allows selective infection or transduction of NK cells. In certain embodiments, the viral glycoprotein allows selective infection or transduction of macrophages. In certain embodiments, the viral glycoprotein allows selective infection or transduction of dendritic cells.

In certain embodiments, the heterologous viral glycoprotein is derived from the glycoprotein of the measles virus, such as the Edmonton measles virus. In certain embodiments, the heterologous viral glycoprotein is derived from the measles virus glycoproteins hemagglutinin (H), fusion protein (F), or both (see, e.g., Frecha et al., Blood. 112:4843-52, 2008; and Frecha et al., Blood. 114:3173-80, 2009, each of which is incorporated by reference in its entirety). In certain embodiments, the viral vector is pseudotyped with a vesicular stomatitis virus G-protein (VSV-G) envelope protein.

In certain embodiments, the viral vector encodes for a protein or nucleic acid (e.g., RNA) of interest. Cells transduced with the viral vector will thereby express the protein or nucleic acid of interest. In certain embodiments, the protein of interest is selected from the group consisting of an antibody or fragment thereof, a hormone, an enzyme, a secreted protein, or a nuclease.

In certain embodiments, the protein of interest influences the regulation of B cells, for example but not limited to promoting cell division, promoting differentiation into different B lineages, inactivating or killing cells, or regulates production or activity of other introduced DNA elements.

In certain embodiments, the secreted protein is a cytokine. In certain embodiments, the cytokine is selected from the group consisting of IL-1, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, secreted form of the p28 subunit of IL27, IL-28, IL-29, IL-30, IL-31, IL-32, IL-33, IL-34, IL-35, IFN-γ, IFN-α, IFN-β and IFN-ω.

In certain embodiments, the cytokine is a chemokine, such as the C type chemokines XCL1 and XCL2, C-C type chemokines (including CCL1-CCL28) and CXC type chemokines (including CXCL1-CXCL17).

In certain embodiments, the protein of interest is a member of the TNF superfamily (e.g., TNF-α, 4-1BB ligand, B cell activating factor, FAS ligand, Lymphotoxin, OX40L, RANKL, and TRAIL).

In certain embodiments, the protein of interest induces immunological tolerance. In this regard, the protein of interest may comprise an IgG-antigen fusion protein (see e.g., Cellular Immunology 235(1), 2005, 12-20).

In certain embodiments, the protein of interest promotes differentiation of a B cell into an antibody secreting cell and/or promote the longevity of the antibody producing cell. Such factors include, for example, Blimp-1, TRF4, anti-apoptotic factors like Bcl-xl or Bcl5, constitutively active mutants of the CD40 receptor. Further proteins of interest encode factors which promote the expression of downstream signaling molecules, such as TNF receptor-associated factors (TRAFs). In this regard, cell activation, cell survival, and antiapoptotic functions of the TNF receptor superfamily are mostly mediated by TRAF1-6 (see e.g., R. H. Arch, et al., Genes Dev. 12 (1998), pp. 2821-2830). Downstream effectors of TRAF signaling include transcription factors in the NF-κB and AP-1 family which can turn on genes involved in various aspects of cellular and immune functions. Further, the activation of NF-κB and AP-1 has been shown to provide cells protection from apoptosis via the transcription of antiapoptotic genes.

In certain embodiments, the protein of interest is an enzyme. In certain embodiments, the enzyme is associated with a lysosomal storage disorder. In one embodiment, the enzyme is iduronidase (IDUA) for treatment or prevention of mucopolysaccharidosis type I (MPS I). In one embodiment, the enzyme is idursulfase for treatment or prevention of mucopolysaccharidosis type II (MPS II). In one embodiment, the enzyme is galsulfase for treatment or prevention of mucopolysaccharidosis type VI (MPS VI). In one embodiment, the enzyme is elosulfase alfa for treatment or prevention of mucopolysaccharidosis type IVA (MPS IV A). In one embodiment, the enzyme is agalsidase beta for treatment or prevention of Fabry's disease. In one embodiment, the enzyme is agalsidase alpha for treatment or prevention of Fabry's disease. In one embodiment, the enzyme is alpha-1-anti-trypsin for treatment or prevention of Alpha-1-anti-trypsin deficiency. In one embodiment, the enzyme is alpha-N-acetylglucosaminidase for treatment or prevention of mucopolysaccharidosis type IIIB (MPS IIIB). In another embodiment, the enzyme is factor VII for treatment or prevention of hemophilia. In one embodiment, the enzyme is lecithin-cholesterol acyltransferase (LCAT) useful for treatment or prevention of, e.g., LCAT deficiency and atherosclerosis. In another embodiment, the enzyme is Apolipoprotein A-1 Milano (ApoA-1 Milano) for treatment or prevention of cardiovascular diseases and disorders, such as, e.g., atherosclerosis. In one embodiment, the enzyme is lipoprotein lipase (LPL) for treatment or prevention of LPL deficiency. In yet another embodiment, the enzyme is phenylalanine hydroxylase for treatment or prevention of phenylketonuria (PKU).

In certain embodiments, the protein of interest is a nuclease. In certain embodiments, the nuclease is a genome-editing nuclease. In certain embodiments, the genome-editing nuclease is selected from a zinc finger nuclease (ZFN), a TALEN, a meganuclease, or an RNA-guided nuclease. In certain embodiments, the RNA-guided nuclease is a CRISPR nuclease. In certain embodiments, the CRISPR nuclease is Cas9. In certain embodiments, the CRISPR nuclease is Cas12 (i.e., Cpf1).

In certain embodiments, the viral vector encodes for a nucleic acid of interest. In certain embodiments, the nucleic acid of interest is an mRNA, an shRNA, a siRNA, a miRNA, an antisense oligonucleotide, or a guide RNA (gRNA). In certain embodiments, the nucleic acid of interest is a gRNA. In certain embodiments, the viral vector encodes for a CRISPR nuclease and one or more gRNAs.

In certain embodiments, the viral vector encodes for Activation-induced cytidine deaminase (AID). In certain embodiments, AID is overexpressed in a human naïve B cell to increase or enhance somatic hypermutation of antibodies.

In certain embodiments, the viral vector encodes for a CRISPR enzyme and a gRNA that targets the AID gene. In certain embodiments, the AID gene is knocked out to prevent or reduce class switching of antibodies.

In certain embodiments, the viral vector encodes for a CRISPR enzyme and a gRNA that targets the FUT8 gene. In certain embodiments, the FUT8 gene is knocked out to prevent or reduce fucosylation of antibodies.

In certain embodiments, the viral vector encodes for a CRISPR enzyme and a gRNA for the integration of a heterologous nucleic acid sequence that encodes a monoclonal antibody (e.g., rituximab, VRCO1).

The following non-limiting examples are provided to further illustrate the present disclosure.

### EXAMPLES

### Example 1—Treatment of B Cells with Statins and Viral Transduction

### Materials and Methods

### Production of Lentivirus:

In a 6-well dish, HEK 293T cells were transfected with 900 ng psPAX2 (addgene plasmid #12260), 100 ng pCMV-VSV-G (addgene plasmid #8454)), and 1000 ng lentiviral transfer plasmid (either GFP+ or GFP− viruses) in the presence of 6 pg polyethylenimine. 48 hours after transfection, supernatant was harvested and filter through a 0.45-micron syringe filter.

### Culture and Transduction of Human Naïve B Cells:

Human naïve B cells were isolated from frozen PBMCs using an EasySep™ human naïve B cell negative selection kit (StemCell catalog number: 17254). Prior to transduction, naïve B cells were co-cultured for 48 hours on stromal feeder cells expressing CD40 ligand. The following modifications were made to the standard co-culture protocol outlined in Su et al. (J. Immunology. 197:4163-4176. 2016). Before plating the feeder cells, a 6-well dish was coated with sterile poly-1-lysine (Sigma Catalog Number: P4707-50 mL), feeder cells were then plated at a density of 1×10⁵ cells per well and allowed to grow overnight. The next day, naïve B cells were isolated and added to the culture at a density of 3×10⁵ cells per well. The culture media (RPMI (hyclone), 10% FBS (hyclone), 100 U/mL penicillin, 100 U/mL streptomycin, 1 mM sodium pyruvate, 10 mM HEPES, 1% MEM nonessential amino acids (All GIBCO)) was supplemented with a recombinant human cytokine mix: IL-2 (50 ng/ml, PreproTech), IL-4 (10 ng/ml, GIBCO), IL-21(10 ng/ml, GIBCO), and BAFF (10 ng/ml, PreproTech); and rosuvastatin (0.5 μM, 5 μM, or 20 μM, Cayman Chemicals catalog number: 12029).

To transduce the naïve B cells, B cells were washed away from the feeder cell layer using cold phosphate buffered saline (PBS). Cells were then pelleted by centrifugation at 400×g for 8 min, and resuspended in viral supernatant supplemented with the recombinant human cytokine mix and 8 μg/mL polybrene. Cells were then spinoculated in a 24-well plate by centrifugation at 1,000×g, 31° C. for 90 min, and then allowed to rest for four hours before being resuspended and plated on fresh feeder cells. 48 hours after transduction, GFP expression was measured by flow cytometry.

### Flow Cytometry:

Cells were blocked with human and mouse Fc block (both BD biosciences) for 15 minutes at room temperature, then stained with anti-human CD19 (Biolegend catalog number: 363029), anti-human IgM (Biolegend catalog number: 314511), and anti-human CD154 (Biolegend catalog number: 310823) in PBS+2% FBS for 20 minutes on ice. Fluorescence was detected on a Macsquant X. Flow cytometry data were analyzed using FlowJo 10.6.

### Results

Naïve B cells were incubated without a stain or with rosuvastatin at a dose of 0.5 μM, 5 μM, or 20 μM. Following incubation, the B cells were transduced with a GFP-expressing lentiviral vector and expression was monitored by flow cytometry, as described above.

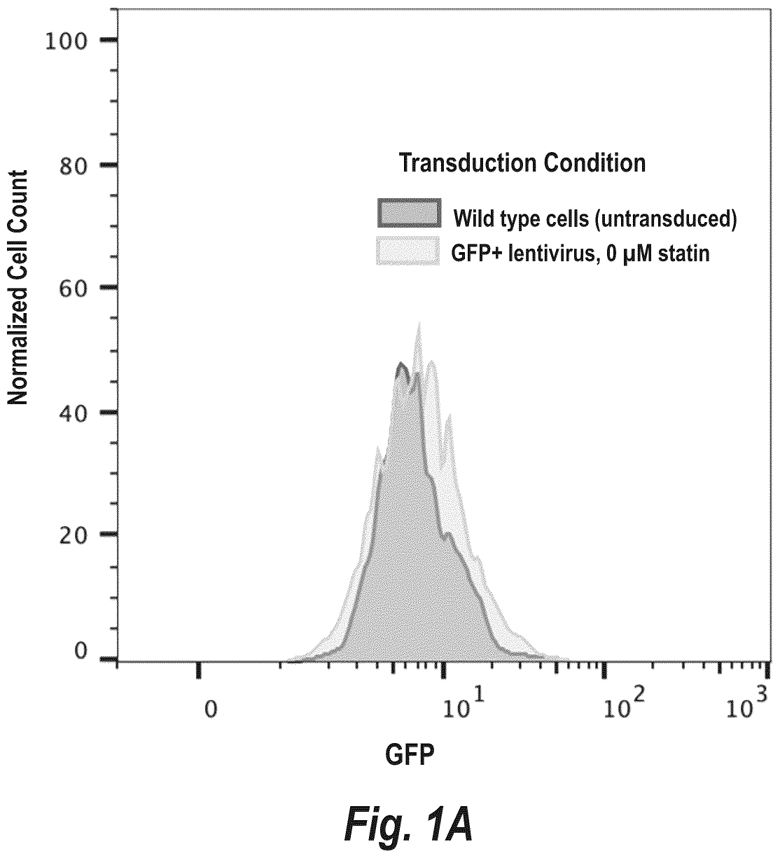

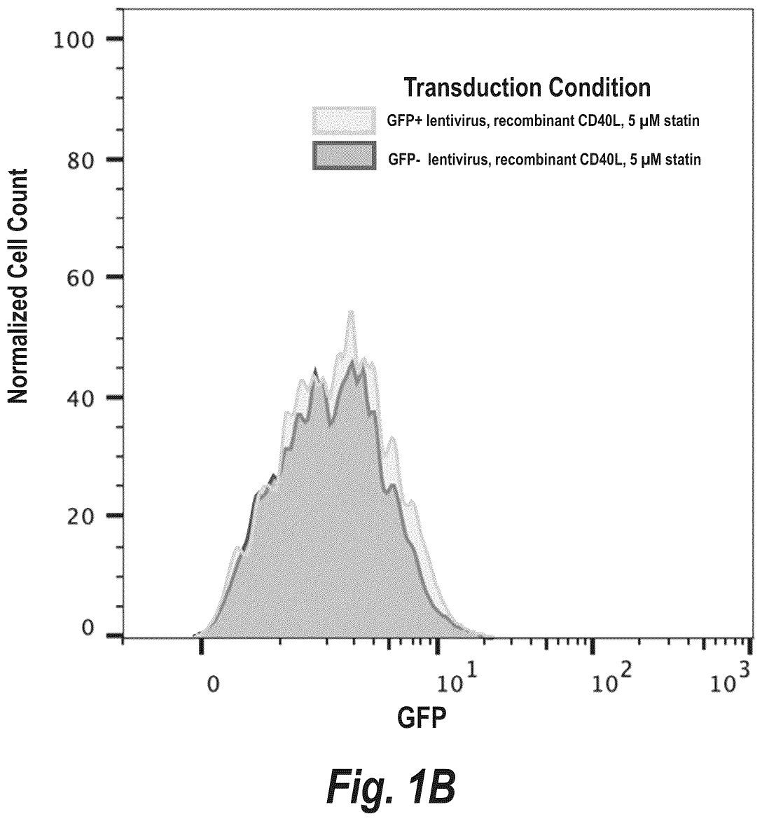

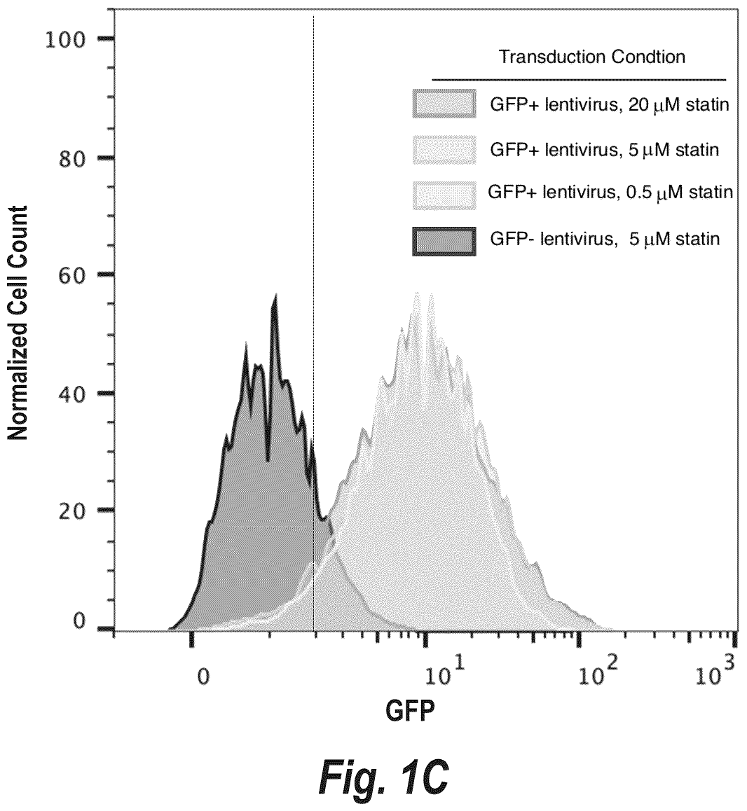

It was surprisingly discovered that B cells incubated with rosuvastatin in culture with CD40L expressing feeder cells at all doses tested were transduced about 100-fold more than B cells that were not incubated with rosuvastatin (FIG. 1A-C). While only about 5% of B cells were transduced without rosuvastatin, over 80% of B cells were transduced with rosuvastatin at all doses tested. This result was notable, because B cells and primary cells are more difficult transduce than cell lines (Gong et al. Molecular Therapy: Methods & Clinical Development. 17: 634-646. 2020). Use of statins represents a useful method for enhancing primary B cell transduction for therapeutic purposes, both ex vivo and in vivo.

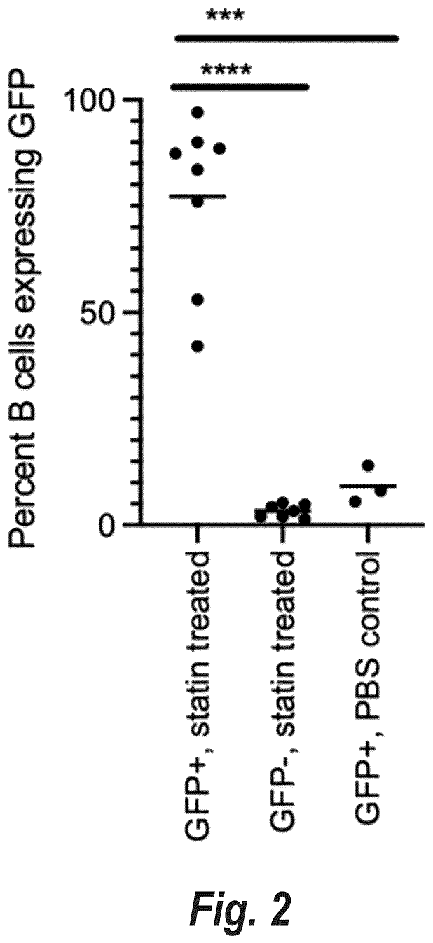

These results were demonstrated again in a similar experiment. Human naïve B cells were isolated from PBMCs using the EasySep Human Naïve B cell isolation kit. 3×10⁵ B cells were then plated in a 6 well dish that had been seeded the day before with 5×10⁴ feeder cells (MS5 mouse stromal cells modified to express human CD40L) or with soluble recombinant CD40L, MEGACD40L® (Enzo biosciences). Cells were then grown in RPMI (hyclone), 10% FBS (hyclone), 100 U/mL penicillin, 100 U/mL streptomycin, 1 mM sodium pyruvate, 10 mM HEPES, 1% MEM nonessential amino acids (All GIBCO)) was supplemented with a recombinant human cytokine mix: IL-2 (50 ng/ml, PreproTech), IL-4 (10 ng/ml, GIBCO), IL-21(10 ng/ml, GIBCO), and BAFF (10 ng/ml, PreproTech) with or without 5 μM rosuvastatin for 48 hours. Cells were washed from a single well with cold PBS, pelleted by centrifugation at 400×g for 8 min, and resuspended in viral supernatant supplemented with the recombinant human cytokine mix and 8 μg/mL polybrene (estimated B cell MOI of 3). Cells were then spinoculated in a 24 well plate by centrifugation at 1,000×g, 31° C. for 90 min, and then allowed to rest for four hours before being resuspended and plated on fresh feeder cells. 48 hours after transduction, GFP expression was measured by flow cytometry, as shown in Table 2. As shown in FIG. 2, GFP expression was substantially higher in B cells treated with a statin compared to B cells treated with the statin and not transduced, or B cells not treated with the statin. Between technical replicates, the average B cell transduction percentage as measured by GFP expression was 80%, with some replicates exceeding 90%.

TABLE 2
Experiment conditions and results

| | Normalized |
|---|---|
| | median |
| | fluorescence |
| Experiment condition | intensity |
| Wild type cells (not transduced) | 1 |
| Soluble recombinant CD40L + 5 μM Statin | 1.1 |
| Feeder culture without statin or increased IL-2 | 1.1 |
| Feeder culture + 0.5 μM statin | 8.8 |
| Feeder culture + 5 μM statin | 10.5 |
| Feeder culture + 20 μM statin | 8.9 |

### Example 2—B Cell Activity Following Viral Transduction

While statins and other agents may increase viral transduction efficiency, this increased viral transduction efficiency should not be at the expense of substantially reduced target cell activity and function.

After incubating B cells with one or more transduction efficiency enhancing agents (e.g., statin) for a sufficient amount of time to increase the transduction efficiency of the B cells with a viral vector, the B cells will be stained for class switching to ensure the naïve B cells retain this ability.

RNA sequencing of immune cells will also be performed before and after treatment with an agent to increase expression of LDL receptor. Data from this experiment will help define the mechanism of increased transduction efficiency and to confirm cell health.

A variety of immune cells (e.g., B cells, NK cells, T cells, plasmablasts) will be transduced with a variety of viruses, including retroviruses and lentiviruses, to express both proteins and RNA. Examples will include:
1) over expression of Activation-induced cytidine deaminase (AID) in human naïve B cells to favor somatic hypermutation of antibodies;
2) CRISPR-Cas9 mediated knock out of AID in human naïve B cells to prevent or reduce class switching of antibodies;
3) CRISPR-Cas9 mediated knock out of the gene FUT8 in human naïve B cells with to prevent or reduce fucosylation of antibodies; and
4) CRISPR-Cas9 mediated knock in of monoclonal antibodies (e.g., rituximab, VRCO1).

### Example 3—B Cells Retain Antibody Class Switching after Statin Treatment

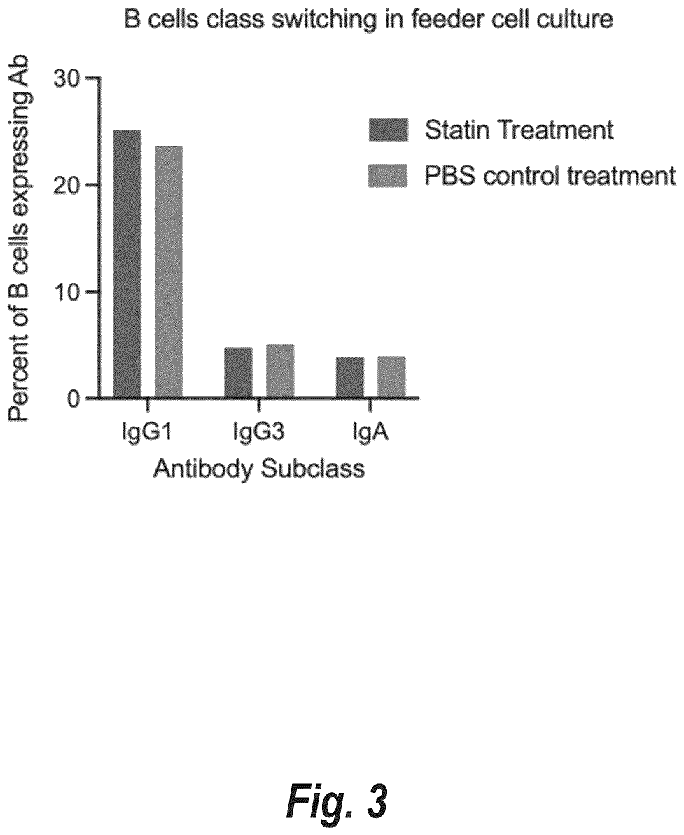

As noted above, statins and other agents should not substantially reduce target cell activity and function. One function of naïve B cells is the ability to engage in antibody class switching. To demonstrate that statin treatment does not impact class switching, freshly isolated naïve B cells (expressing less than 1% IgG1, IgG3, or IgA) were treated with statin or PBS control for 48 hours and then allowed to grow for 14 days in culture with feeder cells and activating cytokine mix. Specifically, human naïve B cells were isolated and cultured as described above in Example 1. Cells were plated at a density of 1×10⁵ cells B cells per 6 well with 1×10⁴ feeder cells plated the previous day. The dual culture was grown in the media and cytokine mix described above with or without 5 μM Rosuvastatin. After 48 hours of treatment, media was changed and cells were allowed to continue growing in the media and fresh cytokines. Every 3-4 days half of the media was removed and replaced with fresh media and cytokines at 1× concentration. Cells were trypsinized and split 1:5 at day 7. Subclass of expressed antibody was assessed by flow cytometry on Day 14. As shown in FIG. 3, addition of the statin did not alter the percent of B cells expressing the measured subclasses of antibody.

### Example 4—LDL Receptor (LDL-R) Antibody Enhancement of Transduction

The effect of using an LDL-R antibody to block LDL-R activity in B cells and feeder cells was tested for effects on transduction. Human naïve B cells were isolated from PBMCs using the EasySep Human Naïve B cell isolation kit. 5×10⁵ cells B cells were then plated in a 6 well dish that had been seeded the day before with 5×10⁴ feeder cells (MS5 mouse stromal cells modified to express human CD40L). Cells were then grown in RPMI (hyclone), 10% FBS (hyclone), 100 U/mL penicillin, 100 U/mL streptomycin, 1 mM sodium pyruvate, 10 mM HEPES, 1% MEM nonessential amino acids (All GIBCO)) was supplemented with a recombinant human cytokine mix: increased IL-2 (250 ng/ml, PreproTech), IL-4 (10 ng/ml, GIBCO), IL-21(10 ng/ml, GIBCO), and BAFF (10 ng/ml, PreproTech) with or without 5 μM rosuvastatin for 48 hours.

Cells were washed from a single well with cold PBS, pelleted by centrifugation at 400×g for 8 min, and resuspended in 50 μL cold PBS and blocked with human and mouse Fc Block for 10 min at room temperature. Cells were then treated with 2 μg/mL of anti-mouse LDL-R antibody for 20 minutes at 4° C. or with PBS. The antibody was Invitrogen LDL-R antibody PA5-46987. After antibody treatment, cells were washed once with cold PBS, then resuspended in 50 μL viral supernatant (GFP expressing virus (GPF+) or a virus that does not encode GFP (GFP−)) supplemented with the recombinant human cytokine mix and 8 μg/mL polybrene (estimated B cell MOI of 3). Cells were spinoculated in a 96 well plate by centrifugation at 1,000×g, 31° C. for 90 min, and then allowed to rest for four hours before being resuspended and plated on fresh feeder cells. 48 hours after transduction, GFP expression was measured by flow cytometry.

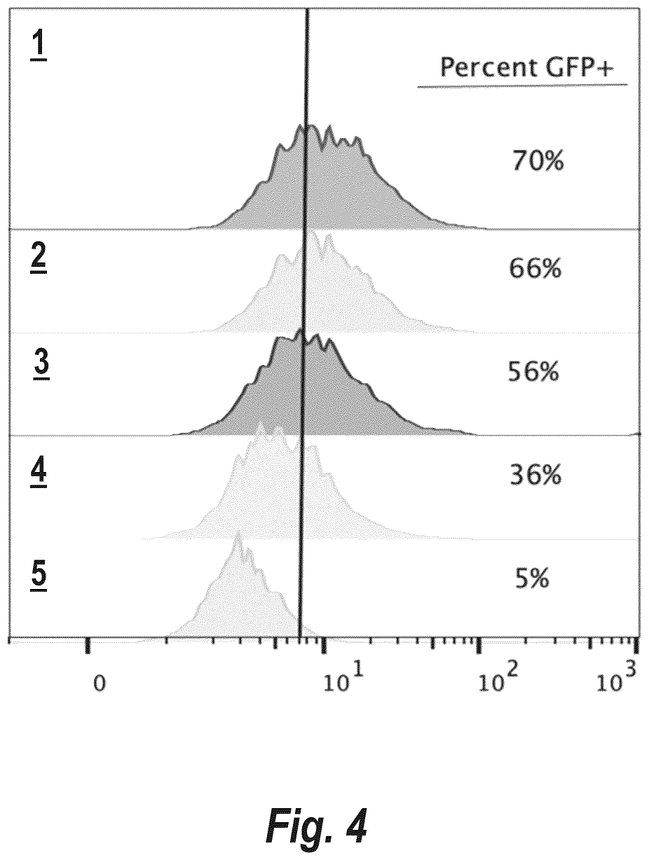

As shown in FIG. 4 and Table 2, addition of an LDL-R antibody to the transduction mix increased viral transduction to human B cells. Moreover, the transduction efficiency increases when the B cells are cultured with feeder cells that express CD40L. Under these conditions, the feeder cells are also transduced. It is hypothesized that the feeder cells are therefore taking up a portion of the virus and thus pre-incubating the dual culture with an LDL-R antagonist, such as an LDL-R antibody, may serve as a blocking agent of the feeder cells.

TABLE 2
Experimental conditions and results

| | | 48 hour treatment | Feeder cell | anti-mouse | Median |
|---|---|---|---|---|---|
| Experiment | | with 5 μM | culture + 250 | LDL-R | Fluorescence |
| ID | Virus | Rosuvastatin | ng/ml (5x) IL-2 | antibody | Intensity |
| 1 | GFP+ | + | + | + | 10 |
| 2 | GFP+ | + | + | − | 9.5 |
| 3 | GFP+ | − | + | + | 8 |
| 4 | GFP+ | − | + | − | 5.5 |
| 5 | GFP− | + | + | − | 3 |

1. A method of increasing transduction efficiency of cells with a viral vector, comprising incubating the cells with one or more transduction efficiency enhancing agents for a sufficient amount of time to increase the transduction efficiency of cells with the viral vector, wherein the one or more transduction efficiency enhancing agents are selected from the group consisting of an AKT inhibitor, a HMG-CoA reductase inhibitor, a LDL-R inhibitor, a CD40 ligand (CD40L), and combinations thereof.

2-13. (canceled)

14. The method of claim 1, wherein the cells are primary cells and the primary cells are co-incubated with one or more cytokines prior to or simultaneously with the one or more transduction efficiency enhancing agents.

15. (canceled)

16. The method of claim 14, wherein the one or more cytokines comprise IL-2, IL-4, IL-21, B-cell activating factor (BAFF), or a combination thereof and wherein the primary cells are co-incubated with:
IL-2 at a concentration of about 5 ng/mL to about 100 ng/mL;
IL-4 at a concentration of about 0.1 ng/mL to about 50 ng/mL;
IL-21 at a concentration of about 0.1 ng/mL to about 50 ng/mL; and/or
BAFF at a concentration of about 0.1 ng/mL to about 50 ng/mL.

17-27. (canceled)

28. The method of claim 1, wherein the one or more transduction efficiency enhancing agents is an AKT inhibitor and the AKT inhibitor comprises ARQ 092, ARQ 751, AT7867, AT13148, A-674563, BAY1125976, capivasertib (also known as AZD5363), GSK690693, GSK2110183, ipatasertib (also known as GDC-0068), LY2780301, miransertib, MK-2206, PF-04691502, triciribine, or a combination thereof.

29. (canceled)

30. The method of claim 1, wherein the one or more transduction efficiency enhancing agents is a HMG-CoA reductase inhibitor and the HMG-CoA reductase inhibitor comprises rosuvastatin, atorvastatin, fluvastatin, lovastatin, pitavastatin, pravastatin, simvastatin, or a combination thereof.

31. (canceled)

32. (canceled)

33. The method of claim 1, wherein the one or more transduction efficiency enhancing agents is CD40L and wherein CD40L is provided by a population of stromal feeder cells that express one or both of a cytokine and CD40L or as isolated CD40L.

34. (canceled)

35. The method of claim 1, wherein the one or more transduction efficiency enhancing agents is an LDL-R inhibitor and the LDL-R inhibitor is an anti-LDL-R antibody or fragment thereof.

36-47. (canceled)

48. A population of cells prepared according to the method of claim 1.

49. A pharmaceutical composition comprising the population of cells of claim 48.

50. A method of transducing a population of cells comprising the steps of:
a) contacting the population of cells with one or more transduction efficiency enhancing agents; and
b) transducing the population of cells with a viral vector;
wherein transduction efficiency is increased in comparison to transduction in the absence of the one or more transduction efficiency enhancing agents, wherein the one or more transduction efficiency enhancing agents are selected from the group consisting of an AKT inhibitor, a HMG-CoA reductase inhibitor, a LDL-R inhibitor, a CD40 ligand (CD40L), and combinations thereof.

51. (canceled)

52. The method of claim 50, wherein the cells are selected from the group consisting of: T cells, B cells, plasmablasts, Natural Killer (NK) cells, macrophages, and dendritic cells.

53-75. (canceled)

76. The method of claim 50, wherein the one or more transduction efficiency enhancing agents is an AKT inhibitor and the AKT inhibitor comprises ARQ 092, ARQ 751, AT7867, AT13148, A-674563, BAY1125976, capivasertib (also known as AZD5363), GSK690693, GSK2110183, ipatasertib (also known as GDC-0068), LY2780301, miransertib, MK-2206, PF-04691502, triciribine, or a combination thereof.

77. (canceled)

78. The method of claim 50, wherein the one or more transduction efficiency enhancing agents is a HMG-CoA reductase inhibitor and the HMG-CoA reductase inhibitor comprises rosuvastatin, atorvastatin, fluvastatin, lovastatin, pitavastatin, pravastatin, simvastatin, or a combination thereof.

79. (canceled)

80. (canceled)

81. The method of claim 50, wherein the one or more transduction efficiency enhancing agents is CD40L and wherein CD40L is provided by a population of stromal feeder cells that express one or both of a cytokine and CD40L or as isolated CD40L.

82. (canceled)

83. The method of claim 50, wherein the one or more transduction efficiency enhancing agents is an LDL-R inhibitor and the LDL-R inhibitor is an anti-LDL-R antibody or fragment thereof.

84-96. (canceled)

97. A population of cells prepared according to the method of claim 50.

98. A pharmaceutical composition comprising the population of cells of claim 97.

99. A method of transducing a population of primary B cells comprising the steps of:
a) contacting the population of primary B cells with one or more statins; and
b) transducing the population of primary B cells with a lentiviral vector;
wherein transduction efficiency is increased in comparison to transduction in the absence of the one or more statins.

100-103. (canceled)

104. The method of claim 99, wherein:
the primary B cells are human naïve B cells;
the statin comprises rosuvastatin at a concentration of about 0.5 μM to about 50 μM; and
the lentiviral vector is pseudotyped with a VSV-G envelope protein.

105. (canceled)

106. A composition comprising a population of primary cells, one or more statins, and a population of feeder cells.

107. The composition of claim 106, further comprising one or more LDL-R inhibitors.

108. (canceled)

109. The composition of claim 106, wherein the feeder cells are stromal feeder cells that express one or both of a cytokine and a CD40 ligand (CD40L).

110. (canceled)

111. (canceled)

112. The composition of claim 109, wherein the stromal feeder cells express IL-2, IL-21, and CD40L.
